(19) United States
(12) Patent Application Publication
(10) Pub. No.: US 2006/0293231 A1
(43) Pub. Date: Dec. 28, 2006

(54) Method for enhancing bone formation

(76) Inventors: Regina Landesberg, Stamford, CT (US); David J. Pinsky, Ann Arbor, MI (US); Ronald W. Katz, Fayetteville, NY (US)

Correspondence Address: COOPER & DUNHAM, LLP, 1185 AVENUE OF THE AMERICAS, NEW YORK, NY, 10036, US

(21) Appl. No.: 11/193,198

(22) Filed: Jul. 29, 2005

Related U.S. Application Data
(60) Provisional application No. 60/592,512, filed on Jul. 30, 2004.

(51) Int. Cl. A61K 38/39; A61K 38/18

(52) U.S. Cl. 514/12

(57) ABSTRACT

This invention provides a method for facilitating bone formation in a subject comprising delivering to a bone formation-requiring site a composition of matter comprising platelet-rich plasma, calcium, a PAR-activating agent and a bone forming material. This invention further provides a method for facilitating bone formation in a subject comprising (a) delivering to a bone formation-requiring site in the subject a composition of matter comprising platelet-rich plasma, calcium and a bone-forming material, and (b) contacting the composition so delivered with a PAR-activating agent other than thrombin. This invention further provides a method for facilitating clot formation in platelet-rich plasma with a PAR-activating agent other than thrombin. This invention further provides a method of producing a formable gel comprising the step of admixing platelet-rich plasma, calcium, a bone-forming material and a PAR-activating agent other than thrombin. Finally, this invention provides related compositions of matter and articles of manufacture.

This application claims the benefit of U.S. Provisional Application No. 60/592,512, filed Jul. 30, 2004, the contents of which are incorporated herein by reference into the subject application.

Throughout this invention, various publications are referred to by Arabic numerals within parentheses. Full citations for these publications are presented immediately before the claims. Disclosures of these publications in their entireties are hereby incorporated by reference into this application in order to more fully describe the state of the art to which this invention pertains.

### BACKGROUND OF THE INVENTION

Platelet-rich plasma (“PRP”) is derived from plasma enriched for platelets and may be efficacious in enhancing wound healing and increasing the rate of bone graft healing in the field of oral and maxillofacial surgery (1, 2). Platelets are known to contain a number of growth factors such as platelet-derived growth factor (“PDGF”), transforming growth factor beta (“TGFβ”), insulin-like growth factors (“IGFs”), epidermal growth factor (“EGF”), and epithelial cell growth factor (“ECGF”) (3, 4).

In the early stages of wound healing following bone fractures or surgical interventions, platelets are activated by the coagulation cascade, particularly thrombin and subendothelial collagen. Activated platelets subsequently release the content of their granules into the wound site. Current methods of PRP preparation use bovine thrombin for clotting, which has been associated with the formation of antibodies to clotting factors V, XI and thrombin, resulting in life-threatening coagulopathies (5). Thrombin is a serine protease mediated through activation of specific thrombin receptors to elicit a variety of cellular responses. The thrombin receptors from human platelets have been sequenced and cloned. Thrombin receptors belong to the seven-transmembrane-spanning domain receptor family coupled to G-proteins (FIG. 1). Thrombin binds to and cleaves its receptor between amino acid residues Arg⁴¹ and Ser⁴² to generate a new amino terminus. The newly generated N-terminal segment of a 14-amino acid peptide SFLLRNPDNKYEPF functions as a “tethered ligand” and activates the receptor (6). Thrombin receptor activator peptide-6 SFLLRN (“TRAP”) is a synthetic peptide corresponding to the amino terminal peptide sequence (amino acids 42-47 of the thrombin receptor) that mimics thrombin in eliciting thrombin-signaled cell responses in platelets independent of receptor cleavage (7, 8).

Although the bone remodeling cascade is not yet fully understood, the sequence of events appears to be under the control of a number of growth factors. Initially, chemotaxis of osteoblast precursors to the site of bone regeneration is mediated by structural proteins such as collagen and/or osteocalcin, as well as growth factors such as PDGF and TGFβ (9, 10, 11). This is followed by proliferation of osteoblasts. PDGF, TGFβ, as well as fibroblast growth factor (“FGF”) and IGF-I and II have all been shown to stimulate proliferation of osteoblasts (12). The differentiation of osteoblasts into mature bone cells is also controlled by growth factors, most significantly by IGF-1 and the bone morphogenetic proteins (“BMPs”) (13, 14).

The aforementioned growth factors within the granules are believed to mediate normal bone healing and regeneration. The efficiency of growth factors in enhancing bone regeneration is likely dependent on dosage, spatial distribution, and temporal sequencing of the available growth factors. Previously reported methods of PRP preparation have reported platelet enrichments of 300 to 700% (3, 4) while assays for growth factors in PRP showed a 7-fold increase in TGFβ and a 30-fold increase of PDGF using Enzyme-Linked Immunosorbent Assay (“ELISA”) (15). Whether these enhanced levels of growth factors in PRP are locally available to the osteoblast at the critical time has not been investigated.

Growth factors activated at the appropriate temporal sequence and spatial distribution have a profound effect on bone regeneration. PDGF has been shown to stimulate mitogenesis and proliferation of mesenchymal-derived cells such as osteoblasts in bone healing. TGFβ is a mitogenic and chemotactic factor that induces proliferation and differentiation of mesenchymal cells into osteoblasts (16). In vitro studies showed that the combination of cytokines and growth factors increased osteoblast proliferation and differentiation (17). The spatial and temporal localization of the growth factors is critical in bone cellular growth and differentiation. Although PRP has proven to be effective in enhancing bone graft healing in a limited number of studies, the temporal sequence and levels of growth factors released from the PRP composite have not been well studied.

Bone regeneration requires osteogenic cell source, growth factors and nutrient supplies. PRP alone does not have any osteoconductive or osteoinductive effect on bone regeneration and is usually used in conjunction with bone graft or bone substitute materials. BioOss (Osteohealth, Shirley, N.Y.) is a bone substitute made from bovine bone after removal of all organic materials. The morphological structure of BioOss resembles human cancellous bone. The porous nature of BioOss provides a scaffold for the formation of the new bone (18). AlloGro (Ceramed, Lakewood, Colo.) is demineralized freeze-dried bone allograft (“DFDBA”). DFDBA has been used extensively in bone grafting, as it is known to have osteoinductive characteristics that will enhance bone cell growth (19). 45S5 BioGlass is a melt-derived bioactive glass ceramic. In vitro studies have shown that BioGlass has the ability to stimulate the growth and estrogenic differentiation of human osteoblasts (20).

The dominant mechanism governing growth factor release from the composites of PRP and bone substrate is diffusion, and this process is driven by the local growth factor concentration gradient present at the graft site. In vitro models of growth factor release must take into account several processes, which are unique, in vivo. Specifically, the temporal concentration and spatial distribution of growth factors within the graft site are expected to vary as a function of fluid infiltration during the initial repair response, as well as the subsequent uptake of available growth factors for cellular function during the bone regeneration stage. Reported in vitro growth factor release studies usually adapt either the static or dynamic mode of incubation. In the static mode, no media exchange is performed and concentration values will eventually reach steady state. In the dynamic mode, fresh solution is added periodically to the system to emulate the location changes in growth factor concentration and utilization.

### SUMMARY OF THE INVENTION

This invention provides a method for facilitating bone formation in a subject comprising delivering to a bone formation-requiring site in the subject a composition of matter comprising platelet-rich plasma, calcium, a PAR-activating agent and a bone-forming material (i.e., a bone regeneration-facilitating material), wherein the composition is free of exogenous thrombin, thereby permitting the composition to facilitate bone formation.

This invention further provides a method for facilitating bone formation in a subject comprising (a) delivering to a bone formation-requiring site in the subject a composition of matter comprising platelet-rich plasma, calcium and a bone-forming material, wherein the composition is free of exogenous thrombin, and (b) contacting the composition so delivered with a PAR-activating agent, other than thrombin, under conditions permitting clot formation in the composition, thereby permitting the composition to facilitate bone formation.

This invention further provides a method for facilitating clot formation in platelet-rich plasma comprising the step of contacting the platelet-rich plasma with a PAR-activating agent, other than thrombin, under conditions permitting clot formation, thereby facilitating clot formation in the platelet-rich plasma.

This invention further provides a method for producing a formable gel comprising the step of admixing platelet-rich plasma, calcium, a bone-forming material and a PAR-activating agent, other than thrombin, thereby producing a formable gel.

This invention further provides a composition of matter comprising platelet-rich plasma, calcium, a PAR-activating agent and a bone-forming material, wherein the composition is free of exogenous thrombin.

Finally, this invention provides an article of manufacture comprising a packaging material having therein, in the same or separate compartments, calcium, a PAR-activating agent and a bone-forming material.

### BRIEF DESCRIPTION OF THE FIGURES

FIG. 1 Schematic of the Function of TRAP. Thrombin binds to and cleaves its receptor between amino acid residue Arg⁴¹ and Ser⁴¹ to generate a new amino terminus. The newly generated N-terminal segment of a 14-amino acid peptide SFLLRNPDNKYEPF functions as a “tethered ligand” and activates the receptor. Thrombin receptor activator peptide-6 SFLLRN (TRAP) is a synthetic peptide corresponding to the amino peptide sequence and mimics thrombin in eliciting thrombin-signaled cell responses in platelets.

FIG. 2 Clot diameter and distribution. Differences in clot diameter were observed between the thrombin, TRAP, and PRP Composites AlloGro (AG), BioOss (BO), and BioGlass (BG). Larger and more evenly distributed clots were observed for the PRP composite groups.

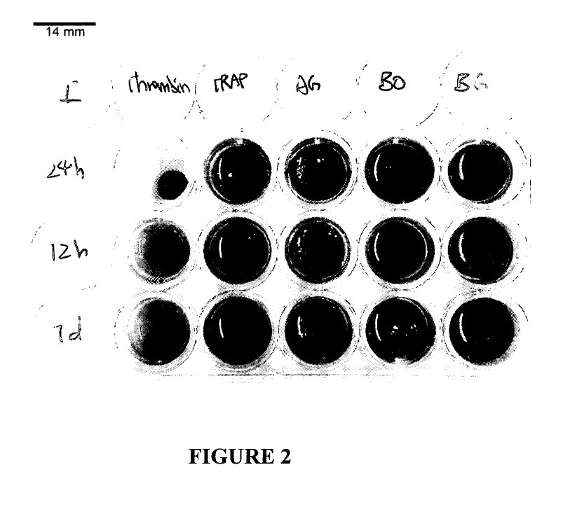

FIG. 3 Temporal Effects of Clotting Substrate on PDGF Release. The thrombin clots released the highest amount of PDGF at 24 hours compared to all other groups tested (p<0.05). Bone substitute groups BioGlass (BG), BioOss, and AlloGro (AG) had approximately 80% less PDGF release than the thrombin group. All bone substitute groups retained on average 60% more PDGF than the thrombin group after 14 days. * denotes statistical significance between groups (p<0.05, n=3).

FIG. 4 Effects of Media Exchange on PDGF Release from Clotting Substrates. In Group 2, media exchange was less frequent compared to Group 1 (see FIG. 3). Similar release profiles were observed for Group 2 substrates when compared to those from Group 1. While the mean values of release may be higher, no statistically significant effects on PDGF release due to the frequency of media change were observed for the Thrombin, TRAP, AlloGro (AG) or BioOss groups.

FIG. 5 Effects of Clotting Substrate on TGFβ Release. The thrombin group released the highest amount of TGFβ post clotting (p<0.05, n=3), with over 81.4% of the growth factor already released from the thrombin clot within 24 hours. The TRAP alone as well as the PRP composite groups released significantly lower levels of TGFβ (p<0.05). Within the bone substrate groups, BG had the highest TGFβ retention compared to the two other groups tested (p<0.05). No significant differences in growth factor release were observed between the bone substrates at the remaining time points. After 14 days, all of the TGFβ in thrombin clots had been released, while all of the bone substrates retained approximately 44% more of the factor compared to the thrombin group. * denotes statistical significance between groups (p<0.05, n=3).

FIG. 6 Effects of Media Exchange on PDGF Release from PRP Composites. A significant difference in PDGF release due to media exchange was only observed in the BioGlass (BG) substrate (p<0.05, n=3), where Group 1 released significantly higher amount of the factor compared to Group 2 in which the media was exchanged less frequently. No significant difference in release was observed for thrombin, TRAP, and all other bone substrates tested as a function of media exchange. It is likely that the AlloGro (AG) and BioOss substrates exhibited improved retention of PDGF compared to the BG group. * denotes statistical significance between groups (p<0.05, n=3).

FIG. 7 Effects of Media Exchange on TGFβ Release from PRP Composites. TGFβ release from the substrates was dependent on media exchange in the PRP composite formed with AG (p<0.05, n=3). Group 1 released significantly higher amount of the factor compared to Group 2, where the media was exchanged less frequently. Media exchange was found to have no significant effect on the retention of TGFβ by the BioOss and BioGlass group. * denotes statistical significance between groups (p<0.05, n=3). These data suggest that BioOss and BioGlass have enhanced retention of TGFβ.

FIG. 8 Additional schematic of the function of thrombin receptor agonist peptide-6 (TRAP). See FIG. 1 for details.

FIG. 9 The addition of thrombin resulted in rapid clotting of platelet-rich plasma (PRP). When 30 units of thrombin was added to 0.5 mL of PRP mixture, complete polymerization occurred at 6 minutes. The addition of 100 units (units thrombin/mL PRP used clinically) resulted in clot formation at 3.25 minutes. The addition of thrombin receptor agonist peptide-6 (TRAP) at 100 μmol/L took 9.25 minutes for the clot to completely solidify.

FIG. 10 The platelet-rich plasma (PRP) control clot (calcium only) took significantly longer to gel and resulted in a clot with very poor structural integrity (data not shown). Virtually all of the clot retraction was complete by 2 hours. Thrombin caused considerable clot retraction. The addition of 100 units of thrombin showed 43% shrinkage of the clot at all time points. In contrast, thrombin receptor agonist peptide-6 (TRAP) (50 and 100 μmol/L) measured only a 15% decrease in the clot diameter

FIG. 11 Clotting times using the different bone substitutes were determined using 100 pmol/L of thrombin receptor agonist peptide-6 (TRAP) with either 25 mg of Allogro, BioOss, or 45S5 Bioactive Glass (BG). Arrows indicate clot size.

FIG. 12 Thrombin receptor agonist peptide-6 (TRAP)/Allogro (11%, 25 mg at 24 hours), TRAP/BioOss (21%, 25 mg at 24 hours), and TRAP/BioGlass (8%, 25 mg at 24 hours) all had significantly less clot retraction than thrombin (56%, 100 units, 24 hours].

FIG. 13 The highest volume of supernatant was collected at day 1. At all time points, the thrombin group measured the largest volume. At day 1, the thrombin group released the highest amount of platelet-derived growth factor (PDGF)-AB (P<0.05), approximately 36% more than the thrombin receptor agonist peptide-6 (TRAP) group. At 7 days, the TRAP group had the highest PDGF-AB release compared with thrombin. At 14 days, both groups released minimal amounts of growth factor (A). The amount of transforming growth factor-β (TGFβ) released by the platelet-rich plasma (PRP)-clotted thrombin and TRAP is shown (B). Similar to the case with PDGF-AB, the thrombin group released the highest amount of TGFβ at day 1 postclotting. The total amount of TGFβ contained in the original PRP volume was measured to be 13,982±2,673.81 pg. In the thrombin group, over 81.4% of the growth factor was already released from the clot in 24 hours. In contrast, the TRAP group released significantly lower levels of TGFβ (P<0.05). Compared with the thrombin group, clotting of PRP with TRAP retained 39.2% more of the growth factor at 72 hours. After 14 days, all of the TGFβ in thrombin clots had been released.

### DETAILED DESCRIPTION OF THE INVENTION

Definitions

“Autologous” shall mean, with respect to any of the instant methods, originating from the subject on whom the instant method is being practiced.

“Bone regeneration-facilitating material” shall mean a solid material which, when placed in, or in juxtaposition to, living bone under suitable conditions, serves as a scaffold for the formation of new bone by bone-forming cells. Bone-forming material includes, without limitation, collagen, bioglass (e.g., 45S5 BioGlass), BioOss (calcium phosphate-based bone graft substitute), Pepgen P-15 (synthetic P-15 peptide bound to a natural form of hydroxylapatite) and AlloGraft (demineralized bone matrix, allograft-based bone graft substitute).

“Bone formation-requiring site” shall mean a site on or in the bone of a subject where the formation of bone is desired. A bone formation-requiring site includes, for example, a space or recess formed in bone through decay or surgical bone removal. Such site can exist on or in any bone (e.g., maxillofacial or vertebral) in any subject.

“Calcium”, with regard to its use in the instant invention, shall mean calcium ions, which exist together with one or more types of negative ions. In one embodiment, calcium exists in the form of a CaCl₂ solution.

“Conditions permitting clot formation” include, without limitation, the presence of calcium ions and a temperature of about 37° C.

“Added growth factor” shall mean a growth factor which does not originate from the platelet-rich plasma used in the instant invention. For example, human PDGF added to human platelet-rich plasma constitutes exogenous growth factor, as opposed to the PDGF already in (i.e., originating from and hence endogenous) the platelet-rich plasma.

“Added thrombin” shall mean thrombin which does not originate from the platelet-rich plasma used in the instant invention.

“Facilitating”, with respect to bone formation, shall mean permitting and/or increasing the rate of bone formation.

“PAR” shall mean thrombin-binding, G protein-coupled protease-activated receptor whose amino terminus is cleaved by thrombin.

“PAR-activating agent” shall mean an agent which binds to PAR, resulting in its activation in the form of a transmembrane signal.

“Platelet-rich plasma,” also referred to in the art as “PRP,” shall mean plasma having therein platelets at a concentration which exceeds the concentration of platelets usually found in whole plasma (i.e., plasma whose components have not been altered, diminished or removed). In one embodiment, platelet-rich plasma has a platelet concentration of between about 300% and 700% greater than the concentration of platelets in whole plasma.

“Subject” shall mean any organism including, without limitation, a mammal such as a mouse, a rat, a dog, a guinea pig, a ferret, a rabbit and a primate. In the preferred embodiment, the subject is a human being.

“Trap-6”, also referred to as “TRAP-6” and “TRAP”, shall mean thrombin receptor activator peptide-6 having the amino acid sequence SFLLRN.

### EMBODIMENTS OF THE INVENTION Embodiments of the Invention

This invention provides a method for facilitating bone formation in a subject comprising delivering to a bone formation-requiring site in the subject a composition of matter comprising (i) autologous platelet-rich plasma, (ii) calcium and (iii) a PAR-activating agent, wherein the composition is free of added thrombin, thereby permitting the composition to facilitate bone formation. In one embodiment, the PAR-activating agent is TRAP-6. In another embodiment, the composition of matter further comprises a bone regeneration-facilitating material. In another embodiment, the bone regeneration-facilitating material is osteoconductive. In another embodiment, the bone regeneration-facilitating material is osteoinductive. In another embodiment, the bone regeneration-facilitating material is selected from the group consisting of collagen, BioOss, PepGen P-15, AlloGro, 45S5 BioGlass and autologous bone. In another embodiment, the composition further comprises one or more added growth factors. In another embodiment, the added growth factor is endogenous to the subject's platelet-rich plasma. In another embodiment, the added growth factor is exogenous to the subject's platelet-rich plasma. In another embodiment, the added growth factor is selected from the group consisting of platelet-derived growth factor, bone morphogenetic protein, transforming growth factor beta, insulin-like growth factor, epidermal growth factor, epithelial cell growth factor, and vascular endothelial growth factor. In another embodiment, the composition is a formable gel. In another embodiment, the subject is human.

This invention further provides a method for facilitating bone formation in a subject comprising (a) delivering to a bone formation-requiring site in the subject a composition of matter comprising (i) autologous platelet-rich plasma and (ii) calcium, wherein the composition is free of added thrombin, and (b) contacting the composition so delivered with a PAR-activating agent, other than thrombin, under conditions permitting clot formation in the composition, thereby permitting the composition to facilitate bone formation. In one embodiment, the PAR-activating agent is TRAP-6. In another embodiment, the composition of matter further comprises a bone regeneration-facilitating material. In another embodiment, the bone regeneration-facilitating material is osteoconductive. In another embodiment, the bone regeneration-facilitating material is osteoinductive. In another embodiment, the bone regeneration-facilitating material is selected from the group consisting of collagen, BioOss, PepGen P-15, AlloGro, 45S5 BioGlass, and autologous bone. In another embodiment, the composition further comprises one or more added growth factors. In another embodiment, the added growth factor is endogenous to the subject's platelet-rich plasma. In another embodiment, the added growth factor is exogenous to the subject's platelet-rich plasma. In another embodiment, the added growth factor is selected from the group consisting of platelet-derived growth factor, bone morphogenetic protein, transforming growth factor beta, insulin-like growth factor, epidermal growth factor, epithelial cell growth factor, and vascular endothelial growth factor. In another embodiment, the subject is human.

This invention further provides a method for facilitating clot formation in platelet-rich plasma comprising the step of contacting the platelet-rich plasma with a PAR-activating agent, other than thrombin, under conditions permitting clot formation, thereby facilitating clot formation in the platelet-rich plasma. In one embodiment, the PAR-activating agent is TRAP-6. In another embodiment, the platelet-rich plasma is admixed with a bone regeneration-facilitating material. In another embodiment, the bone regeneration-facilitating material is osteoconductive. In another embodiment, the bone regeneration-facilitating material is osteoinductive. In another embodiment, the bone regeneration-facilitating material is selected from the group consisting of collagen, BioOss, PepGen P-15, AlloGro and 45S5 BioGlass, and autologous bone. In another embodiment, the platelet-rich plasma is human platelet-rich plasma.

This invention further provides a method for producing a formable gel comprising the step of admixing platelet-rich plasma, calcium and a PAR-activating agent, other than thrombin, so as to permit clot formation, thereby producing a formable gel. In one embodiment, the PAR-activating agent is TRAP-6. In another embodiment, the method further comprises the step of admixing a bone regeneration-facilitating material with the platelet-rich plasma, calcium and PAR-activating agent. In another embodiment, the bone regeneration-facilitating material is osteoconductive. In another embodiment, the bone regeneration-facilitating material is osteoinductive. In another embodiment, the bone regeneration-facilitating material is selected from the group consisting of collagen, BioOss, PepGen P-15, AlloGro, 45S5 BioGlass, and autologous bone. In another embodiment, the method further comprises admixing one or more growth factors with the platelet-rich plasma, calcium, a bone regeneration-facilitating material and a PAR-activating agent. In another embodiment, the growth factor is selected from the group consisting of platelet-derived growth factor, bone morphogenetic protein, transforming growth factor beta, insulin-like growth factor, epidermal growth factor, epithelial cell growth factor and vascular endothelial growth factor. In another embodiment, the platelet-rich plasma is human platelet-rich plasma.

This invention further provides a composition of matter comprising platelet-rich plasma, calcium and a PAR-activating agent, wherein the composition is free of added thrombin. In one embodiment, the PAR-activating agent is TRAP-6. In another embodiment, the composition further comprises a bone regeneration-facilitating material. In another embodiment, the bone regeneration-facilitating material is osteoconductive. In another embodiment, the bone regeneration-facilitating material is osteoinductive. In another embodiment, the bone regeneration-facilitating material is selected from the group consisting of collagen, BioOss, PepGen P-15, AlloGro, 45S5 BioGlass, and autologous bone. In another embodiment, the composition further comprises one or more added growth factors. In another embodiment, the added growth factor is endogenous to the platelet-rich plasma. In another embodiment, the added growth factor is exogenous to the platelet-rich plasma. In another embodiment, the added growth factor is selected from the group consisting of platelet-derived growth factor, bone morphogenetic protein, transforming growth factor beta, insulin-like growth factor, epidermal growth factor, epithelial cell growth factor and vascular endothelial growth factor. In another embodiment, the platelet-rich plasma is human platelet-rich plasma.

This invention further provides an article of manufacture comprising a packaging material having therein, in the same or separate compartments, calcium and a PAR-activating agent. In one embodiment, the PAR-activating agent is TRAP-6. In another embodiment, the article comprises in the same or separate compartments, a bone regeneration-facilitating material, calcium and a PAR-activating agent. In another embodiment, the bone regeneration-facilitating material is osteoconductive. In another embodiment, the bone regeneration-facilitating material is osteoinductive. In another embodiment, the bone regeneration-facilitating material is selected from the group consisting of collagen, BioOss, PepGen P-15, AlloGro, 45S5 BioGlass, and autologous bone. In another embodiment, the article further comprises one or more added growth factors. In another embodiment, the added growth factor is endogenous to platelet-rich plasma. In another embodiment, the added growth factor is exogenous to platelet-rich plasma. In another embodiment, the added growth factor is selected from the group consisting of platelet-derived growth factor, bone morphogenetic protein, transforming growth factor beta, insulin-like growth factor, epidermal growth factor, epithelial cell growth factor and vascular endothelial growth factor. In another embodiment, the article further comprises instructions for use in facilitating bone formation in a subject. In another embodiment, the article further comprises container(s) and reagent(s) for preparing platelet-rich plasma and, using the platelet-rich plasma so prepared, admixing the platelet-rich plasma with the calcium and a PAR-activating agent to form a bone-formation-enhancing composition.

This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

### EXAMPLE 1

Synopsis

Preparation of PRP with thrombin results in a large immediate release of growth factor into the supernatant, which could be lost into the interstitium in vivo. Materials other than thrombin such as TRAP and bone substitutes are believed to be more efficacious in sustaining growth factor levels critical for the cascade of events leading to bone formation. Growth factor retention was a function of both the substrate used as well as the specific growth factor examined. Use of this in vitro system to control growth factor release from PRP composites has use in enhancing bone regeneration.

Materials and Methods
Preparation of PRP

PRP was prepared by a modification of Landesberg et al (4). Sixty milliliters of venous blood from healthy adult volunteers were mixed with ACD Solution B in 9.0 ml vacutainer tubes (Becton Dickinson, Franklin Lakes, N.J.). The ACD solution contained 13.2 g/L trisodium citrate, 4.8 g/L citric acid, and 14.7 g/L dextrose. The samples were centrifuged at 200×g for 15 minutes (ACE Surgical Supply Company, Inc; Brockton, Mass.). The plasma and buffy coat layers were removed and placed into 5 ml tubes, and tubes were spun at 200×g for another 10 minutes. The upper half of the preparation was designated platelet-poor plasma (PPP) and subsequently discarded. The lower half of the plasma and the pellet were re-suspended and pooled to be the platelet-rich plasma (PRP).
Preparation of PRP-Bone Substitute Composites

PRP composites were prepared by mixing PRP and TRAP with bone substitutes commonly utilized in the clinical setting. Twenty four-well plates (Corning Inc., Corning, N.Y.) were coated with 1% bovine serum albumin (Sigma, St. Louis, Mo.) and incubated for 1.5 hours at 37° C. Sterilized BioOss (25 mg, 0.5-1.0 mm), AlloGro (25 mg), and 45S5 Bioactive Glass (25 mg, 300 pm) were uniformly dispersed within the wells. Fresh PRP (0.5 ml) aliquots were then dispensed into the pre-coated 24-well plates, and 30 μl of 10% calcium chloride solution were added to each well. Experiments were performed in triplicate (n=3) with the addition of bovine thrombin (75 units) or thrombin receptor activating peptide (TRAP, H₂N-Ser-Phe-Leu-Leu Arg-Asn-NH₂). TRAP was used to clot PRP for all bone substitute groups examined.
Growth Factor Release—Effects of Bone Substitute and Incubation Mode

The temporal release of growth factors was examined as a function of bone substitute and mode of sample incubation. Specifically, the PRP composites were allowed to clot, and all samples were incubated at 37° C. and humidified environment for up to 14 days. The media were exchanged for all groups at 1 day after incubation. The volume of fluid released from the clot was measured and equal volume of fresh Dulbecco's Modification of Eagle's Medium (DMEM, Mediatech, Herdon, Va.) without serum was added back to each well. Subsequently, in the dynamic incubation mode (Group 1), the fluid released from the clot was collected and equal volume of fresh DMEM was added back into the well at 3, 7, and 14 days. In the static mode (Group 2), the media was exchanged only at the designated time points (7 and 14 days). All collected supernatant samples were stored at −70° C. prior to analyses.
Quantification of PDGF and TGFβ

Supernatants collected from all time points for both Group 1 and 2 were assayed for PDGF-AB and TGFβ content using diagnostic kits from R & D Systems (Minneapolis, Minn.). Both assays are based on a sandwich enzyme immunoassay technique. The PDGF-AB assay uses a pre-coated microtiter plate with a monoclonal antibody to PDGF-AA. Preparation and dilution of samples and standards were performed as directed by the manufacturer. Both the standards and the samples were incubated for 3 hours at room temperature. The plate was washed with buffer and a conjugated antibody to PDGF-BB was added to the wells and incubated at room temperature for an additional 1 hour. The plate was then washed and substrate added for 20 minutes at room temperature. The reaction was stopped and absorbance was determined at 450 nm using a spectrophotometer (SpectraFluor Plus, Tecan, Maennedorf, Switzerland). A standard curve was generated and the PDGF-AB levels (pg/ml) of each sample were determined. The total amount of growth factor was calculated based on the amount of supernatant obtained after clot retraction.

TGFβ was assayed with a similar enzyme immunoassay technique. A dilution series of TGFβ standards was prepared in 100 μl volumes in 96-well microtiter plates coated with TGFβ receptor Type II. The sample supernatants (0.025 ml) obtained from PRP composites were diluted with 0.075 ml of phosphate buffer saline solution. The samples were then activated with 0.1 ml of 1.0 N HCl incubated at room temperature for 10 minutes, neutralized by an addition of 0.1 ml of 1.2 N NaOH/0.5 M HEPES (N-[2-hydroxyethyl] piperazine-N-[2-ethanesulfonic acid]). The supernatant fractions were then incubated for 3 hours at room temperature. The wells were then washed and enzyme-conjugated polyclonal antibody to TGFβ1 was added and allowed to incubate for 1.5 hours at room temperature. The reaction was stopped and absorbance was measured at 450 nm using a spectrophotometer (SpectraFluor Plus, Tecan, Maennedorf, Switzerland). A standard curve was generated and the TGFβ levels (pg/ml) of each sample were determined. The total amount of growth factors was calculated based on the amount of supernatant obtained after clot retraction.
Statistical Analyses

All results were expressed as mean±standard deviation. Multi-way Analysis of Variance (ANOVA) was performed and the Tukey-Kramer test was used to compare between the means. Significance was determined at p<0.05.

Results

Thrombin results in rapid clotting of PRP. When 30 units of thrombin are added to 0.5 ml of PRP mixture, complete polymerization occurs at 6 min. Addition of 100 units (similar to the ratio of that used clinically) and 300 units resulted in clot formation at 1.25 min. The addition of TRAP at 100 μm took 14.5 min for the clot to completely solidify. The addition of collagen accelerated the clot formation by 4-7 min.

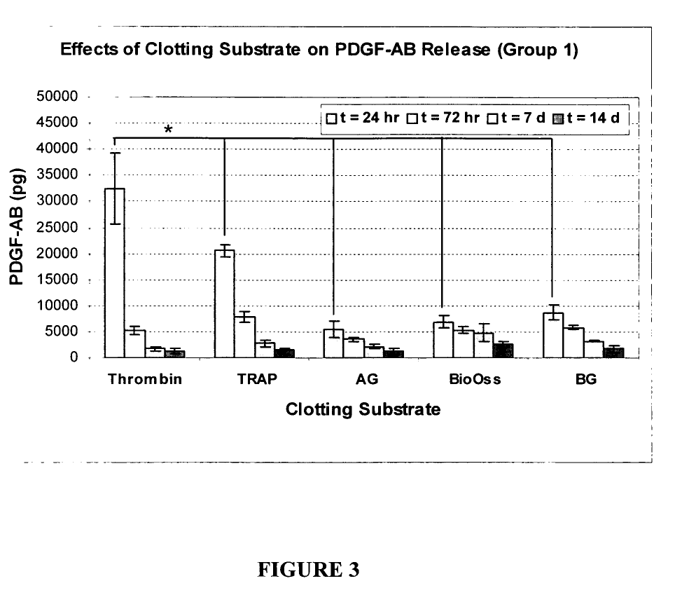

The highest volume of supernatant was collected for all groups at day 1. At all time points the thrombin group measured the largest volume. At day 1 the thrombin group released the highest amount of PDGF-AB (p<0.05, 32,526 pg), approximately 36% more than the TRAP group (20,642 pg). PDGF-AB release from the bone substitute groups, BioGlass (8,757 pg), BioOss (6,847 pg), and AlloGro (5,519 pg) was approximately 80% less than that from the thrombin group (FIG. 3). At 7 days the TRAP group had the highest PDGF-AB release (11,624 pg) compared to thrombin (5,068 pg), while BioGlass (5,304 pg), BioOss (6,646 pg), and AlloGro (3,775 pg) measured similar levels. At 14 days all groups released minimal amounts of growth factor. All bone substitute groups retained on average 60% more PDGF-AB than the thrombin group after 14 days.

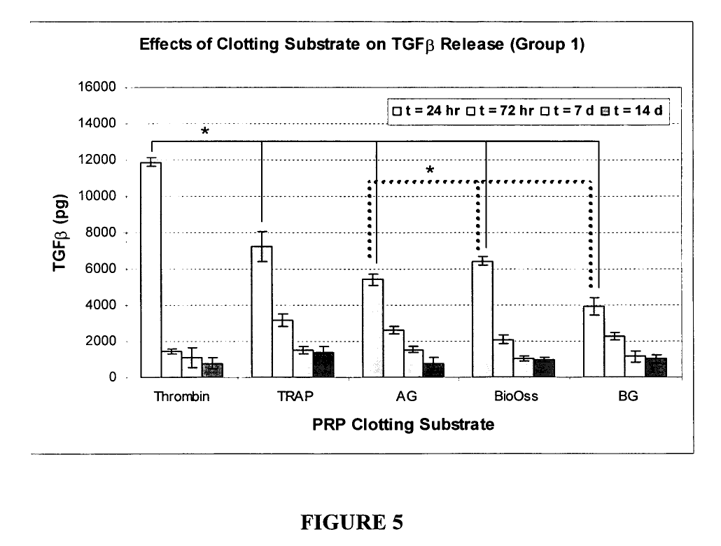

The amount of TGFβ released by the PRP composite is shown in FIG. 5. Similar to the case with PDGF-AB, the thrombin group released the highest amount of TGFβ at day 1 post clotting. The total amount of TGFβ contained in the original PRP volume was measured to be 13,982±2,673.81 pg.

In the thrombin group (TH), over 81.4% of the growth factor was already released from the clot. In contrast, the TRAP alone as well as the PRP composite groups released significantly lower levels of TGFβ (p<0.05). Compared to the thrombin group, clotting of PRP with TRAP retained 39.2% more of the growth factor, while the bone substrate groups retained significantly higher amounts of TGFβ (AG retained 54.3%, BioOss retained 45.8%, and BG retained 67.0%). Within the bone substitute groups, BG had the highest TGFβ retention compared to the two other groups tested (p<0.05). No significant differences in growth factor release were observed among the bone substrates at the remaining time points. After 14 days, all the TGFβ in thrombin clots was released, while all the bone substrates retained approximately 44% more of this growth factor compared to the thrombin group.

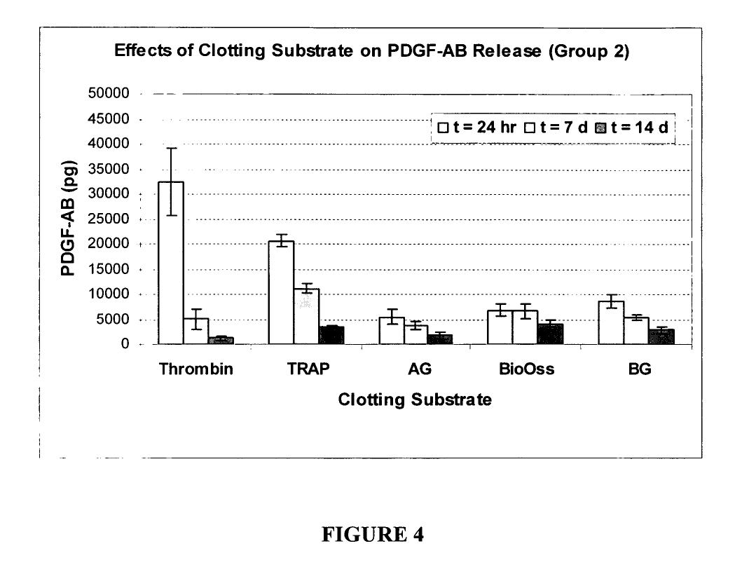

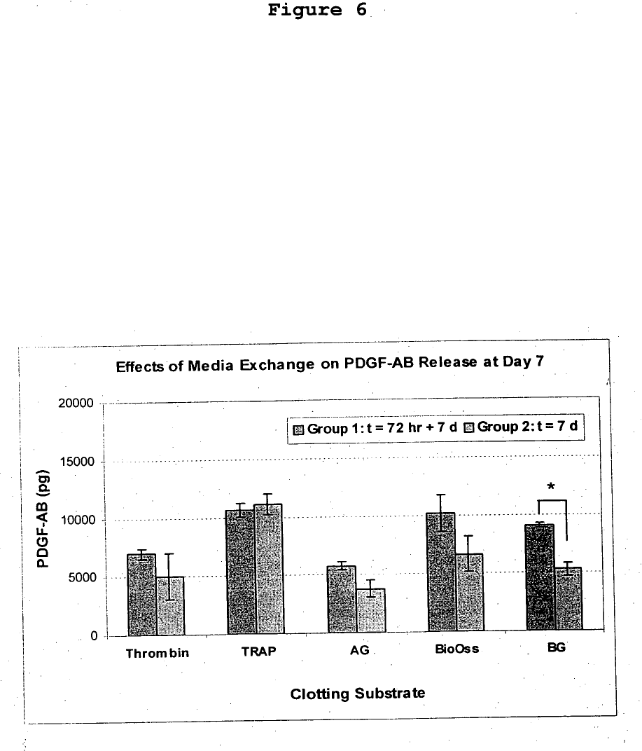

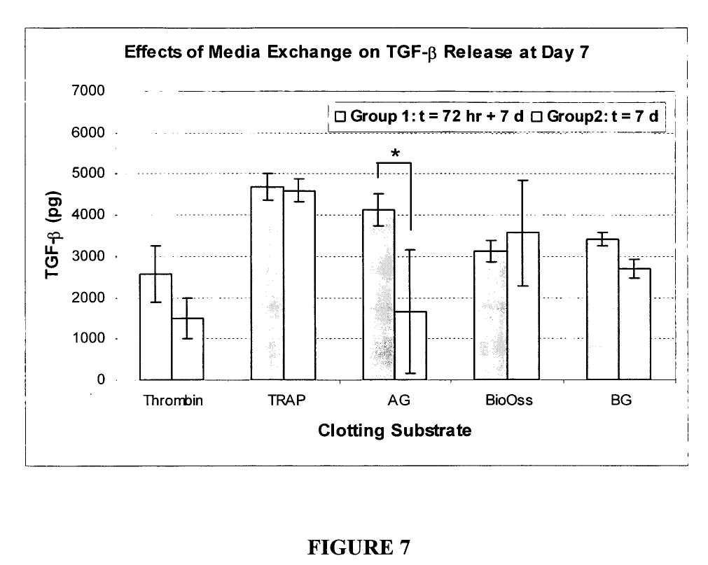

The effects of media exchange on PDGF and TGFβ release are shown in FIGS. 6 and 7, respectively. FIGS. 3 and 4 compare specifically the release profile of PDGF under dynamic (Group 1) versus static (Group 2) modes of media exchange. In terms of PDGF release, a significant difference in release was only observed for the BG substrate as a function of media exchange (p<0.05). Group 1 released higher amounts of PDGF compared to Group 2 (p<0.05, n=3). No significant difference in release was observed for thrombin, TRAP, and all other bone substitutes tested as a function of frequency of media exchange. In contrast, TGFβ release from the substrates was affected by media exchange in the PRP composite formed with AG (p<0.05). Group 1 released significantly higher amount of the factor compared to Group 2, where the media was exchanged less frequently. Media exchange was found to have no significant effect on the retention of either PDGF-AB or TGFβ by the BO group.

Discussion

In this study, the release of growth factors PDGF-AB and TGFβ from different preparations of PRP was evaluated, and the effect of the frequency of media exchange on the release profiles was monitored. Current methods of preparing PRP utilize commercially available thrombin derived from bovine plasma which has been associated with the development of antibodies to clotting factors V, XI, and thrombin, resulting in the risk of life-threatening coagulopathies (5). This invention provides an alternative method for PRP clotting using Thrombin Receptor Activator Peptide-6 (TRAP), a synthetic peptide that mimics thrombin in eliciting thrombin-signaled cell responses in platelets. Studies have shown that TRAP results in significantly less clot retraction than thrombin while providing excellent working time in the preparation of PRP. Since TRAP is a synthesized peptide it is devoid of contaminated coagulation factors present in bovine thrombin, negating the risk of serious coagulopathies. Therefore TRAP was chosen to activate clotting of PRP in the present study. The results from this study show that TRAP, when used alone, retained more PDGF-AB than thrombin. Moreover, when TRAP was combined with BO, BG and AG, approximately 60% more growth factor was retained compared to thrombin. These results suggest that PRP with TRAP and TRAP plus bone substitutes are potentially superior to PRP prepared with thrombin.

In this study PDGF-AB and TGFβ release from PRP and PRP composites were evaluated. There were no significant differences in PDGF-AB release among any of the bone substitutes at all time points. These results suggest that the retention of PDGF-AB by the TRAP/bone substitute composites is relatively nonspecific to the tested substitutes. In contrast, differences in TGFβ release were observed among the various bone substitutes as a function of time. Growth factor retention is dependent on both the substitute used, as well as the specific growth factor examined. In contrast to PDGF-AB, there was a large release of TGFβ (40-50%) at day 1 in all bone substitute groups. This suggests that the bone substitutes have a much greater potential for the retention of PDGF-AB. While IGF-1 was not tested in this study it is known to be a component of PRP and is critical to osteoblast differentiation in the later stages of bone regeneration making it extremely important to delay its release. IGF-1 is considerably smaller than PDGF-AB and TGFβ (PDGF, 30 kD; TGF, 24 kD; IGF-1, 7.6 kD) (21) and may have a different release profile than the growth factors tested.

The effects of media exchange on growth factor release from PRP and the PRP composites were evaluated. The rationale was to identify an optimal and realistic in vitro model that will mimic the local bone grafting environment in which the growth factors will be delivered. PRP-derived growth factors at the grafting site will most likely be released from the platelets and utilized during bone healing. However, continuous interstitial fluid exchange or infiltration of vasculature will alter the local concentration of the growth factors. The frequency of media exchange was used to mimic this continuous process.

In all experiments, PRP clot media was changed at day 1. Group 1 (dynamic mode) had additional media changes at 3 days, while in Group 2 (static mode) no additional changes were performed between 1 day and 7 days. In the static mode, it is anticipated that the growth factor release level would reach steady state and then remain at the same levels. While it was observed that mean value release of PDGF-AB was consistently higher in the group where the media was more frequently exchanged (dynamic mode), the release profile reached a plateau in both cases after 7 days. If growth factor release were based solely on equilibration, it would be expected that the accumulative factor levels from day 1 to day 7 in the dynamic group would be equal to the total amount of growth factor released for the static group within the same period. Interestingly, it was found that PDGF release from the BG sample was lower in Group 2 than in Group 1. In contrast, TGFβ was significantly less in Group 2 of the AG sample, leading us to conclude that AG and BO enhance the retention of PDGF-AB, while BO and BG promote retention of TGF.

In this study, growth factor retention by a select group of bone substitutes was examined. In terms of substrate chemistry, AlloGro is based on demineralized bone matrix, which is composed of organics, while BioOss is deproteinated bone with a calcium phosphate matrix. Bioactive glass develops a surface calcium phosphate layer which has been shown to promote bone bonding. Calcium phosphate ceramic-based materials have been combined with a variety of growth factors including TGFβ to successfully promote bone healing in vitro and in vivo (23, 24, 25). The specificity or the chemical nature of the interaction between the specific growth factors and the biomaterial substrate tested here remains unclear at this time.

At present, there exist several schools of thought regarding the clinical efficacy of PRP in enhancing bone regeneration in oral and maxillofacial surgery (22, 26). Based on the known sequence of events that takes place during bone regeneration, it is desirable to have PDGF and TGFβ present in the early phases followed by TGFβ during the intermediate phase, and IGF-1 and BMPs during the final phase of bone differentiation and maturation. The observed difference in growth factor retention and temporal availability reported in the present study suggest that the bone regeneration potential of PRP may be controlled by matching growth factor release profiles with the known cascade for bone healing.

### REFERENCES FOR EXAMPLE 1 AND BACKGROUND OF THE INVENTION

1. Whitman, D. H., et al., “Platelet Gel: An autologous alternative to fibrin glue with applications in oral and maxillofacial surgery,” J. Oral Maxillofac. Surg. 55: 1294 (1997).
2. Marx, R. E., et al., “Platelet-rich plasma: growth factor enhancement for bone grafts,” Oral Surg. 85: 638 (1998).
3. Weibrich, G., et al., “Growth factor levels in platelet-rich plasma and correlation with donor age, sex and platelet count,” J. Cranio-Maxillofac. Surg. 30: 97 (2002).
4. Landesberg, R., et al., “Quantification of Growth Factor Levels Using a Simplified Method of Platelet-Rich Plasma Gel Preparation,” J. Oral Maxillofac. Surg. 58: 297 (2000).
5. Zehnder, J. L. and Leung, L. L. K., “Development of antibodies to thrombin and factor V with recurrent bleeding in a patient exposed to topical bovine thrombin,” Blood 76: 2011 (1990).
6. Vu, T. K. H., et al., “Molecular cloning of a functional thrombin receptor reveals a novel proteolytic mechanism of receptor activation,” Cell 64: 1057 (1991).
7. Coughlin, S. R., “Protease-activated receptors and platelet function,” Thromb. Haemost. 82(2): 353 (1999).
8. Stiernberg, J., et al., “The role of thrombin and thrombin receptor activating peptide (TRAP-508) in initiation of tissue repair,” Thromb. Haemost. 70(1): 158 (1993).
9. Mundy, G. R., et al., “Unidirectional migration of osteosarcoma cells with osteoblast characteristics in response to products of bone resorption,” Calcif. Tissue Int. 34: 542 (1982).
10. Mundy, G. R. and Poser, J. W. “Chemotatic activity of the gamma-carboxyglutamic acid containing protein in bone,” Calcif. Tissue Int. 35: 164 (1985).
11. Pfeilschifter, J., et al., “Chemotactic response of osteoblast-like cells to transforming growth factor i,” J. Bone Miner. Res. 5: 825 (1990).
12. Canalis, E., et al., “Skeletal growth factors,” Crit. Rev. Eukaryot. Gene Expr. 3: 155 (1993).
13. Manolagas, S. C., “Birth and death of bone cells: Regulatory mechanisms and implications for the pathogenesis and treatment of osteoporosis,” Endocr. Rev. 21: 15 (2000).
14. Canalis, E., et al., “Growth factors and the regulation of bone remodeling,” J. Clin. Invest. 81: 277 (1989).
15. Babbush, C. A., et al., “An in vitro and in vivo evaluation of autologous platelet concentrate in oral reconstruction,” Implant. Dent. 12(1): 24 (2003).
16. Bonewald, L. F. and Mundy, G. R., “Role of TGF beta in bone remodeling,” Ann. N.Y. Acad. Sci. 593: 91 (1990).
17. Lind, M., “Growth factors: possible new clinical tools. A review,” Acta. Orthop. Scand. 67(4): 407 (1996).
18. Valentini, P., et al., “Sinus grafting with porous bone mineral (Bio-Oss) for implant placement: a 5-year study on 15 patients,” Int. J. Perio. Rest. Dent. 20(3): 245 (2000).
19. Schwartz, Z., et al., “Ability of commercial demineralized freeze-dried bone allograft to induce new bone formation,” J. Periodontol. 67(9): 918 (1996).
20. Xynos, I. D., et al., “Bioglass 45S5 stimulates osteoblast turnover and enhances bone formation In vitro: implications and applications for bone tissue engineering,” Calcif. Tissue Int. 67(4): 321 (2000).
21. Canalis, E., “Primer on the metabolic bone diseases and disorders of mineral metabolism,” Fifth edition: Chapter 4, Osteogenic growth factors, Am. Soc. Bone Min. Res., Washington, D.C., 28, 2003.
22. Marx, R. E., “Platelet-Rich Plasma: Evidence to Support its Use,” J. Oral Maxillofac. Surg. 62: 489-496 (2004).
23. Desilets, C. P., et al., “Development of synthetic bone-repair materials for craniofacial reconstruction,” J. Craniofac. Surg. 1(3): 150-3 (1990).
24. Wiltfang, J., et al., “Sinus floor augmentation with β-tricalcium phosphate (β-TCP): does platelet-rich plasma promote its osseous integration and degradation?” Clin. Oral Impl. Res. 14: 213-218 (2003).
25. Sun, J., et al., “Collagen-hydroxyapatite/Tricalcium Phosphate Microspheres as a Delivery System for Recombinant Human Transforming Growth Factor-b 1,” Artifical Organs, 27(7): 605-612 (2003).
26. Freymiller, E. G. and Aghaloo, T. L., “Platelet-Rich Plasma: Ready or Not,” J. Oral Maxillofac. Surg. 62: 484-488 (2004).

### EXAMPLE 2

Synopsis

The use of platelet-rich plasma (PRP) as an adjunct to bone grafting procedures in oral and maxillofacial surgery has seen an increase in popularity since its introduction in 1997 by Whitman et al (1). PRP has technical benefits and theoretically may enhance wound healing by increasing availability of critical growth factors that are released by platelet degranulation (2). Preparation of PRP requires concentrating platelets through centrifugation and subsequent polymerization to form a semisolid gel. Several commercially available methods for enrichment of platelets are currently used in the clinical setting.

At the present, all methods of PRP gelation use calcium and bovine thrombin to initiate PRP clot-formation. The use of bovine thrombin has unfortunately been associated with the development of antibodies to clotting factors V and XI and thrombin, resulting in the risk of potential life-threatening coagulopathies (3-8). Consequently, there is a growing interest in identifying alternative agents for PRP clotting.

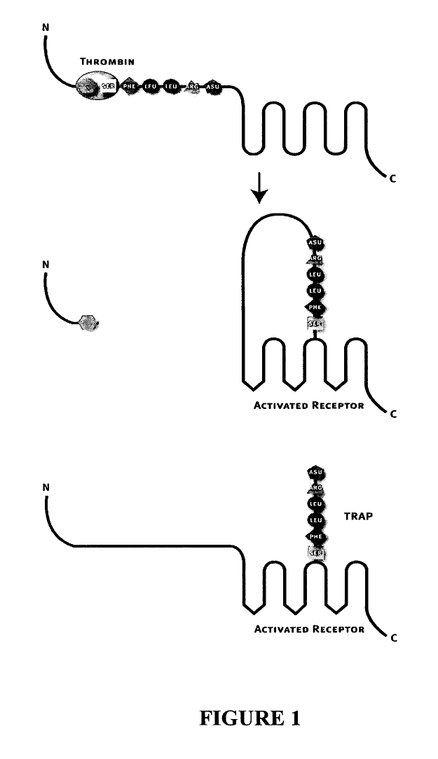

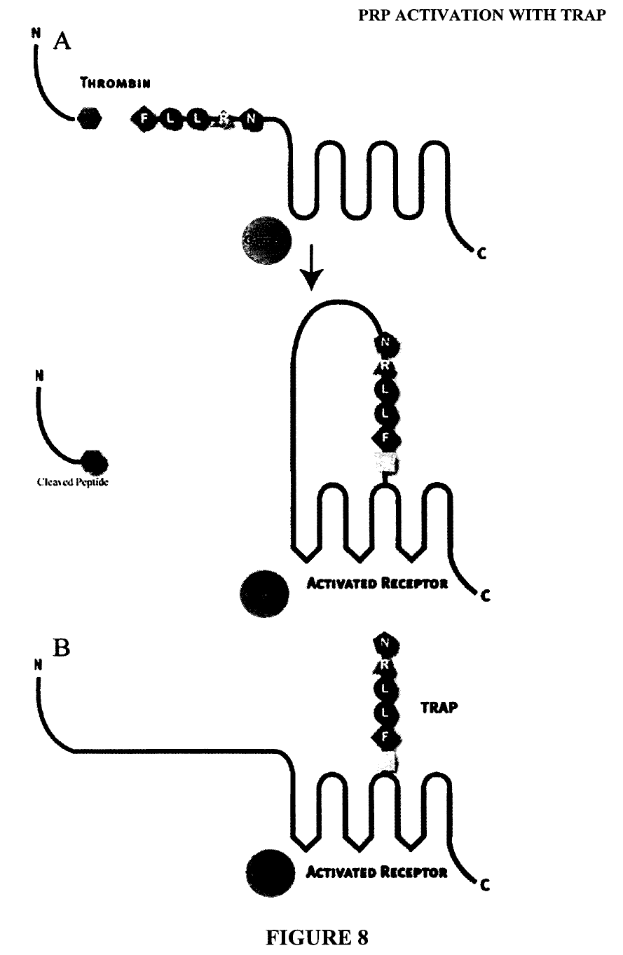

Thrombin signaling of platelets is mediated by a G protein-coupled protease-activated receptor (PAR). The PAR is activated after thrombin binding and subsequent cleavage of the amino-terminal end of the receptor (9). This new amino terminus acts as a tethered ligand and binds intramolecularly to the body of the PAR, resulting in a transmembrane signal (FIG. 8A). In contrast, synthetic peptides such as thrombin receptor agonist peptide-6 (TRAP) activate the receptor independent of receptor cleavage (FIG. 1B). TRAP is a hexapeptide that corresponds to amino acids 42 to 47 of the thrombin receptor and mimics the effects of thrombin such as platelet aggregation, an increase in tyrosine phosphorylation, inhibition of cAMP, and increase in cytosolic calcium (10-12). These reports suggest that TRAP is a promising candidate as a clotting agent for PRP.

After gel formation, PRP undergoes clot retraction, and growth factor such as platelet-derived growth factor (PDGF), transforming growth factory (TGFβ), and vascular endothelial growth factor (VEGF) are released (13, 14). The degree of clot retraction could have significant effects on the bioavailability of these growth factors and consequently the clinical efficacy of PRP-enhanced bone regeneration. In addition, excessive shrinkage of the PRP gel may affect graft adaptation, resulting in significant loss of growth factors from the graft composite. The time course and the amount of shrinkage that takes place after PRP gelation using thrombin or alternative clotting agents have not been fully characterized.

Clinically, PRP is routinely combined with bone substitutes such as BioOss, an inorganic bovine bone substitute, AlloGro, demineralized freeze-dried human bone allograft and 45S5 BioGlass, a melt-derived bioactive glass ceramic, during oral and maxillofacial surgery procedures. BioOss and BioGlass are osteoconductive materials, and Allogro is osteoinductive (15, 16). The present study also determines the potential of TRAP-6 to clot PRP in conjunction with bone substitutes. The time course and the amount of shrinkage that takes place after PRP gelation with TRAP in the presence of bone substitutes are also evaluated.

The objective of this study was to investigate the use of TRAP as an alternative to thrombin in the clotting of PRP. The optimal concentration, the time course of gelation, and the resultant clot retraction were evaluated using an in vitro assay system. The hypothesis is that TRAP will offer a safer alternative to PRP gelation resulting in adequate working time and decreased clot retraction compared with thrombin was tested. Furthermore, it was tested whether TRAP-initiated PRP clot retraction would result in a reduced release of relevant growth factors, potentially increasing the bioavailability of these regenerative agents.

Materials and Methods
Preparation of Platelet-rich Plasma

PRP was prepared through a modification of the met=thod of Landesberg et al (13). Briefly, 60 mL of volunteer blood was collected into 10-mL tubes containing 1.0 mL ACD Solution B (Becton Dickinson, Franklin Lakes, N.J.). The ACD solution contained 13.2 g/L trisodium citrate, 4.8 g/L citric acid, and 14.7 g/L dextrose. The tubes were spun at 200×g for 10 minutes. The plasma and buffy coat layer were transferred to 10-mL tubes and spun at 20×g for 10 minutes. The upper half (platelet-poor plasma) was discarded; the lower (PRP) was resuspended and used for this study.
Clotting of Platelet-Rich Plasma with Thrombin and Thrombin Receptor Agonist Peptide-6

PRP (0.5 mL) aliquots were dispensed into 24-well plates precoated with 1% bovine serum albumin (Sigma, St Louis, Mo.). Thirty microliters of 10% CaCl₂ solution (American Reagent Laboratories, Shirley, N.Y.) were added to each well. Experiments were performed in triplicate (n=3) with the addition of bovine thrombin (30, 100, 300 units; GenTrac, Middleton, Wis.) or TRAP (10, 50, 100 μmol/L; Bachem Bioscience Inc, King of Prussia, Pa.). At 30 minutes, if still attached, the clots were gently released from the sides of the culture well with a pipette tip. PRP prepared with CaCl₂ solution with the addition of thrombin or TRAP served as the control group.

Clotting times were monitored by visualization. Clot retraction was determined by measuring the clot diameter at 1, 2, 4, and 24 hours, and the value was normalized against the well diameter.
Preparation of Platelet-Rich Plasma-Bone Substitute Composites with Thrombin Receptor Agonist Peptide-6

After the optimization of TRAP concentration, experiments were repeated with the addition of 25 or 50 mg of bone substitutes. To minimize nonspecific binding, the culture well (Corning Inc, Corning, N.Y.) was precoated with 1% bovine serum albumin (Sigma) and incubated for 1.5 hours at 37° C. Sterilized BioOss (25 mg, 0.5 to 1.0 mm; Osteohealth, Shirley, N.Y.), AlloGro (25 mg; Ceramed, Lakewood, Colo.), and 45S5 Bioactive Glass (25 mg, 300 μm; Mo-Sci, Rolla, Minn.) were uniformly dispersed within the wells. Fresh PRP (0.5 mL) aliquots were then dispensed into the precoated 24-well plates, and 30 μl of 10% CaCl₂ solution (American Reagent Laboratories, Shirley, N.Y.) was added to each well. Experiments were performed in triplicate (n=3) with the addition of TRAP (H₂N-Ser-Phe-Leu-Leu-Arg-Asn-NH₂) to the wells containing bone substitutes.
Growth Factor Release from Platelet-Rich Plasma Prepared with Thrombin or Thrombin Receptor Agonist Peptide-6

The temporal release of growth factors was examined as a function of time and mode of PRP preparation. Specifically, the PRP clotted with thrombin or TRAP was allowed to gel, and all samples were incubated at 37° C. in a humidified environment for up to 14 days. The volume of fluid released from the clot was measured. Growth factor release was assessed at 1, 3, 7, and 14 days. All collected supernatant samples were stored at −70° C. before analysis.
Quantification of Platelet-Derived Growth Factor and Transforming Growth Factor-β

Supernatants were assayed for PDGF-AB and TGFβ content using diagnostic kits from R & D Systems (Minneapolis, Minn.). Both assays are based on a sandwich enzyme immunoassay technique. The PDGF-AB assay used a precoated microtiter plate with a monoclonal antibody to PDGF-AA. Preparation and dilution of samples and standards were performed as directed by the manufacturer. Both the standards and the samples were incubated for 3 hours at room temperature. The plate was washed with buffer, and a conjugated antibody to PDGF-BB was added to. the wells and incubated at room temperature for 1 additional hour. Following the addition of substrate and termination of the reaction, the absorbance was determined at 450 nm using a spectrophotometer (SPECTRAFluor Plus; Tecan, Maennedorf, Switzerland). A standard curve was generated, and the PDGF-AB level (pg/mL) of each sample was determined. The total amount of growth factors was calculated based on the amount of supernatant obtained after clot retraction.

TGFβ was assayed with a similar enzyme immunoassay technique. A dilution series of TGFβ standards were prepared in 96-well microtiter plates coated with TGFβ receptor Type II. The sample supernatants (0.025 mL) obtained from PRP composites were diluted with 0.075 mL of phosphate-buffered saline solution. The samples were then activated with 0.1 mL of 1.0 N HCl, incubated at room temperature for 10 minutes, and neutralized by an addition of 0.1 mL of 1.2 N NaOH/0.5 mol/L HEPES (N-[2-hydroxyethyl]piperazine-N-[2-ethanesulfonic acid]). The supernatant fractions were then incubated for 3 hours at room temperature. The wells were then washed, and enzyme-conjugated polyclonal antibody to TGFβ1 was added and allowed to incubate for 1.5 hours at room temperature. The reaction was stopped and absorbance was measured at 450 nm using a spectrophotometer (SPECTRAFluor Plus; Tecan). A standard curve was generated, and the TGFβ level (pg/mL) of each sample was determined. The total amount of growth factors was calculated based on the amount of supernatant obtained after clot retraction.
Statistical Analysis

All results were expressed as mean±SD. Multiway analysis of variance (ANOVA) was performed and the Tukey-Kramer test was used to compare between the means. Significance was determined at P<0.05.

Results

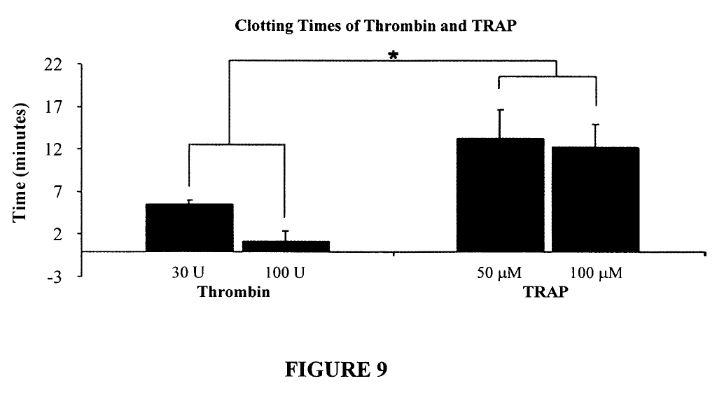

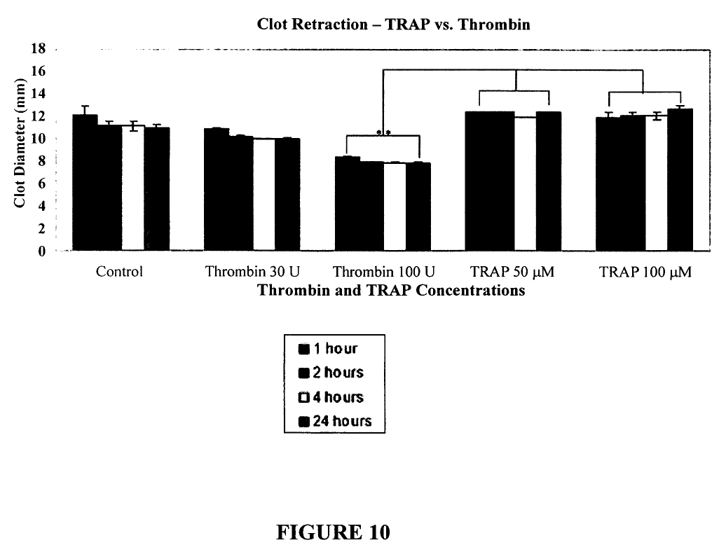

Thrombin resulted in rapid clotting of PRP. When 30 units of thrombin was added to 0.5 mL of PRP mixture, complete polymerization occurred at 6 minutes. Addition of 100 units (units thrombin/mL PRP used clinically) resulted in clot formation at 3.25 minutes. The addition of TRAP at 100 μmol/L took 9.25 minutes for the clot to completely solidify (FIG. 9). The PRP control clot (calcium only) took significantly longer to gel and resulted in a clot with very poor structural integrity (data not shown). Virtually all of the clot retraction in the groups tested was complete by 24 hours. Thrombin caused considerable clot retraction. The addition of 100 units of thrombin led to 43% shrinkage of the clot at all time points, whereas 30 units of thrombin resulted in a 34% clot retraction (FIG. 10). In contrast, TRAP at both 50 and 100 μmol/L concentrations showed only a 15% decrease in the clot diameter (FIG. 10).

Clotting times using the different bone substitutes were determined vising 100 μmol/L of TRAP with 25 mg of Allogro, BioOss, or BioGlass. Clots with Allogro showed complete solidification within 12 minutes, whereas the BioOss (25 mg) composite did not completely polymerize until 13 minutes. The BioGlass group was completely clotted at 8.75 minutes. The clotting time between bone substitutes was not significantly different from each other.

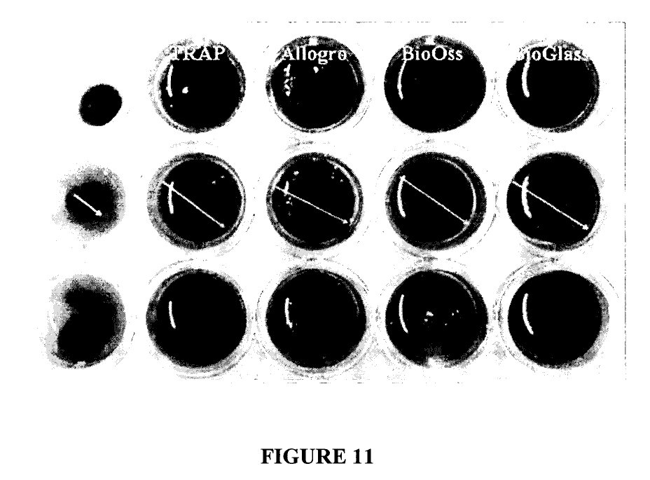

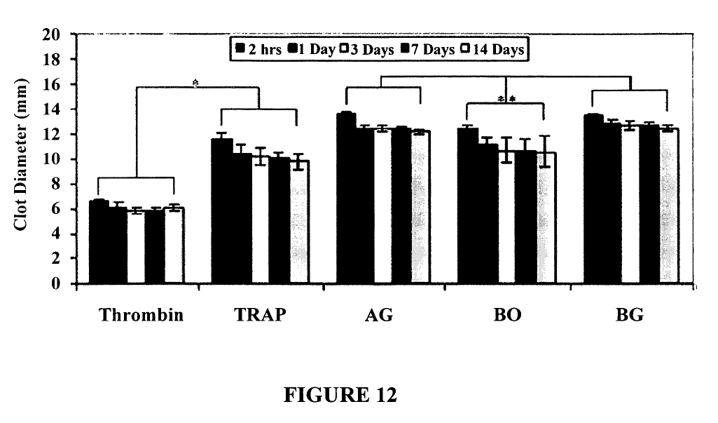

Clot retraction was measured at 2, 24, 72, 168, and 336 hours for all groups with bone substitutes. Retraction was essentially completed by 24 hours, as no significant differences between the 24- and 72-hour measurements were noted for thrombin, TRAP, or TRAP plus bone substitutes. At 24 hours, the TRAP/Allogro (11%±2%), TRAP/BioOss (21%±4%), and TRAP/BioGlass (8%±3%) groups all had significantly less clot retraction than thrombin (56%±3%) (FIGS. 11, 12). Quantitative analyses (FIG. 12) of clot diameter corresponded well with observations (FIG. 11). In addition, the highest volume of supernatant was collected at day 1. At all time points, the thrombin group measured the largest release volume.

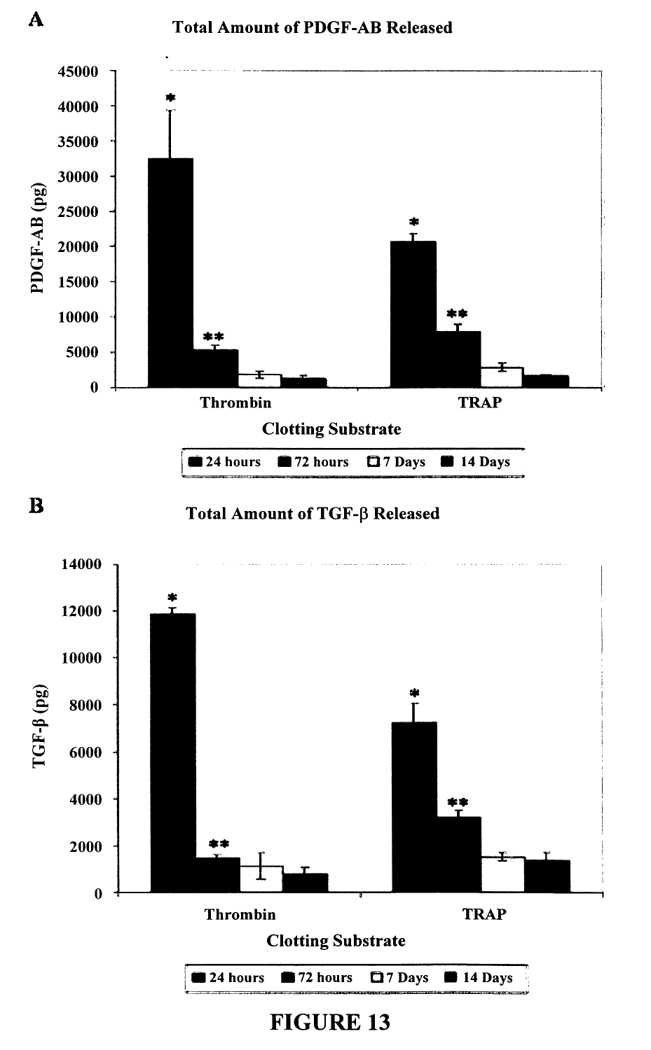

In terms of growth factor release as a function of PRP clotting method, at day 1 the thrombin group released the highest amount of PDGF-AB (P<0.05, 32,526±6,752.4 pg), approximately 36% more than the TRAP group (20,642±1,170.0 pg). At 7 days, the TRAP group had the highest PDGF-AB release (2,797.6±612.0 pg) compared with thrombin (1,738.2 ±443.0 pg) (FIG. 13A). At 14 days, both groups released minimal amounts of growth factor.

The amount of TGFβ released by the PRP composite is presented (FIG. 13B). Similar to the case with PDGF-AB, the thrombin group released the highest amount of TGFβ at day 1 postclotting. The total amount of TGFβ contained in the original PRP volume was measured to be 13,982±2,673.81 pg. In the thrombin group, over 81.4% of the growth factor was already released from the clot within 24 hours. In contrast, the TRAP group released significantly lower levels of TGFβ (P<0.05). Compared with the thrombin group, clotting of PRP with TRAP retained 39.2% more of the growth factor at 72 hours. After 14 days, all of the TGFβ present in the original PRP thrombin clots had been released.

Discussion

A number of centrifugation protocols are presently available for concentrating platelets; however, all methods use bovine thrombin to accelerate clot formation (2, 17). Bovine thrombin has been associated with the formation of antibodies to factors V and XI and thrombin that may result in life-threatening coagulopathies (3-8). This study examined the potential of TRAP as an alternative to thrombin for the clotting of PRP. The findings from this study show that compared with thrombin, TRAP preparations of PRP resulted in longer working time, larger clot diameters, and extended bioavailability of specific growth factors necessary for bone regeneration.

TRAP is a synthetic hexapeptide that activates the thrombin receptor independent of receptor cleavage. It corresponds to amino acids 42 to 47 of the thrombin receptor and mimics the effects of thrombin (9-12). An in vitro system was developed here to quantify polymerization time of PRP using thrombin, TRAP, and TRAP-bone substitutes. When a clinically relevant concentration of thrombin was used to clot PRP, it resulted in rapid clot formation with a large amount of clot retraction. In contrast, at concentrations of 50 and 100 μmol/L, TRAP significantly decreased the degree of clot retraction while providing an appropriate working time. The efficacy of TRAP was unaffected with the addition of several bone substitutes (Allogro, BioOss, and BioGlass), as minimal clot retraction was measured when the clot had fully polymerized. In a related study, it was recently shown that on clot retraction, significant levels of growth factors are released from the PRP gel (18). Consequently, decreasing clot retraction in PRP polymerization will potentially retain optimal growth factor amounts with a desired delay in bioavailability as well as maintenance of graft adaptation to the perimeter of the bone defect. Therefore, one of the goals of this study was to identify a PRP clotting agent that would result in minimal clot retraction.

PRP is derived from plasma enriched with platelets and may be efficacious in enhancing bone regeneration when used in oral and maxillofacial surgical procedures. While the use of PRP in bone grafting procedures offers some mechanical advantage based on the adhesive properties of the gel, significant controversy exists regarding the ability of PRP to accelerate bone regeneration. Marx et al (2) performed radiographic and histomorphometric studies on 88 mandibular discontinuity defects of 5 cm or more, where half of the patients received a cancellous posterior ilial bone graft with PRP. The study found that the PRP grafts matured earlier and had higher total bone content than the grafts without PRP. There have, however, been a number of clinical as well as animal studies that have failed to show the efficacy of PRP in facilitating bone repair (19, 21). It is postulated that the stimulation of bone healing by PRP is due to the increased concentration of relevant growth factors, including PDGF, TGFβ, vascular endothelial growth factor (VEGF), and insulin-like growth factors (IGFs) (2, 14, 22). While the optimal levels as well as the temporal sequence of growth factor delivery in bone regeneration have not been established, growth factors that promote osteoblast differentiation and maturation (ie, TGFβ and IGF) are believed to act at a later time in the bone regeneration cascade (23-25). Within this context, the findings from the present study suggest that the alternate PRP preparation method using TRAP and TRAP/bone substitutes can enhance bone graft integration and maturation by delaying the release of relevant PRP-derived growth factors and extending their bioavailability during the bone regeneration process.

In conclusion, the use of TRAP in the preparation of PRP provides a safe and economical alternative to thrombin while minimizing the amount of clot retraction and the potentially rapid loss of critical bone regenerative growth factors into the interstitium.

### References for Example 2

1. Whitman D H, Berry R L, Green D M: Platelet gel: An autologous alternative to fibrin glue with applications in oral and maxillofacial surgery. J Oral Maxillofac Surg 55:1294, 1997.
2. Marx R E, Carlson E R, Eichstaldt R M, et al: Platelet-rich plasma; growth factor enhancement for bone grafts. Oral Surg 85:638, 1998.
3. Cmolik B L, Spero J A, Magovern G J, et al: Redo cardiac surgery: Late bleeding complications from topical thrombin-induced factor five deficiency. J Thorac Cardiovasc Surg 105:222, 1993.
4. Spero J A: Bovine thrombin-induced inhibitor of factor V and bleeding risk in postoperative neurosurgical patients. Neuro-surgery 78:817, 1993.
5. Muntean W, Zenz W, Finding K, et al: Inhibitor to factor V after exposure to fibrin sealant during cardiac surgery in a two-year-old child. Acta Paediatr 83:84, 1994.
6. Sosolik R C, Theil K S, Brandt J T: Clinical pathology rounds: Anti-bovine thrombin antibody. Lab Med 27:651, 1995.
7. Landesberg R, Moses M, Karpatkin M: Risks of using platelet rich plasma gel. J Oral Maxillofac Surg 56:1116, 1998 (letter).
8. Ortel T L, Mercer M C, Thames E H, et al: Immunologie impactand clinical outcomes after surgical exposure to bovine thrombin. Ann Surg 233:88, 2000.
9. Coughlin S R: Protease-activated receptors and platelet function. Thromb Haemost 82:353, 1999.
10. Coughlin S R: Protease-activated receptors in vascular biology. Thromb Haemost 86:298, 2001.
11. Vassallo R R Jr, Kieber-Emmons T, Cichowski K, et al: Structure-function relationships in the activation of platelet thrombin receptors by receptor-derived peptides. J Biol Chem 267:6081, 1992.
12. Andersen H, Greenberg D L, Fujikawa K, et al: Protease-activated receptor 1 is the primary mediator of thrombin-stimulated platelet procoagulant activity. Proc Natl Acad Sci USA 96:11189, 1996.
13. Landesberg R, Roy M, Glickman R S: Quantification of growth factor levels using a simplified methods method of platelet-rich plasma gel preparation. J Oral Maxillofac Surg 58:297, 2000.
14. Hudson-Goodman P, Girard N, Jones M B: Wound repair and the potential use of growth factors. Heart Lung 19:379, 1990.
15. Ducheyne P: Bioceramics: Material characteristics versus in vivo behavior. J Biomed Mater Res 21:219, 1987.
16. Jensen S S, Aaboe M, Pinholt E M, et al: Tissue reaction and material characteristics of four bone substitutes. Int J Oral Maxillofac Implants 11:55, 1996.
17. Jakse N, TangI S, Gilli R, et al: Influence of PRP on autogenous sinus grafts: An experimental study on sheep. Clin Oral Implant Res 14:578, 2003.
18. Tsay R C, Vo J, Burke A, et al: Differential growth factor retention by platelet-rich plasma composites. Oral Maxillofac Surg 63:521, 2005.
19. Wiltfang J, Kloss F R, Kessler P, et al: Effects of platelet-rich plasma on bone healing in combination with autogenous bone and bone substitutes in critical-size defects. Clin Oral Implant Res 15:187, 2004.
20. Aghaloo T L, Moy P K, Freymiller E G: Investigation of platelet-rich plasma in rabbit cranial defects: A pilot study. J Oral Maxillofac Surg 60:1176, 2002.
21. Butterfield K J, Bennett J, Gronowicz G, et al: Effect of platelet-rich plasma with autogenous bone grafts for sinus augmentation in a rabbit model. J Oral Maxillofac Surg 61:97, 2003 (suppl).
22. Weibrich G, Kleis W K G, Hafner G, et al: Growth factor levels in platelet-rich plasma and correlation with donor age, sex, and platelet count. J Craniomaxillofac Surg 30:97, 2002.
23. Canalis E, Pash J, Varghese S: Skeletal growth factors. Crit Rev Eukaryot Gene Expr 3:155, 1993.
24. Canalis E, McCarthy T, Centrella M: Growth factors and the regulation of bone remodeling. J Clin Invest 81:277, 1989.
25. Canalis E: Osteogenic growth factors, in Primer on the Metabolic Bone Diseases and Disorders of Mineral Metabolism (ed 5). Washington, D.C., American Society of Bone and Mineral Research, 2003, p 28.

1. A method for facilitating bone formation in a subject comprising delivering to a bone formation-requiring site in the subject a composition of matter comprising (i) autologous platelet-rich plasma, (ii) calcium and (iii) a PAR-activating agent, wherein the composition is free of added thrombin, thereby permitting the composition to facilitate bone formation.

2. The method of claim 1, wherein the PAR-activating agent is TRAP-6.

3. The method of claim 1, wherein the composition of matter further comprises a bone regeneration-facilitating material.

4. The method of claim 3, wherein the bone regeneration-facilitating material is osteoconductive.

5. The method of claim 3, wherein the bone regeneration-facilitating material is osteoinductive.

6. The method of claim 3, wherein the bone regeneration-facilitating material is selected from the group consisting of collagen, BioOss, PepGen P-15, AlloGro, 45S5 BioGlass and autologous bone.

7. The method of claim 1, wherein the composition further comprises one or more added growth factors.

8. The method of claim 7, wherein the added growth factor is endogenous to the subject's platelet-rich plasma.

9. The method of claim 7, wherein the added growth factor is exogenous to the subject's platelet-rich plasma.

10. The method of claim 7, wherein the added growth factor is selected from the group consisting of platelet-derived growth factor, bone morphogenetic protein, transforming growth factor beta, insulin-like growth factor, epidermal growth factor, epithelial cell growth factor, and vascular endothelial growth factor.

11. The method of claim 1, wherein the composition is a formable gel.

12. The method of claim 1, wherein the subject is human.

13. A method for facilitating bone formation in a subject comprising (a) delivering to a bone formation-requiring site in the subject a composition of matter comprising (i) autologous platelet-rich plasma and (ii) calcium, wherein the composition is free of added thrombin, and (b) contacting the composition so delivered with a PAR-activating agent, other than thrombin, under conditions permitting clot formation in the composition, thereby permitting the composition to facilitate bone formation.

14-23. (canceled)

24. A method for facilitating clot formation in platelet-rich plasma comprising the step of contacting the platelet-rich plasma with a PAR-activating agent, other than thrombin, under conditions permitting clot formation, thereby facilitating clot formation in the platelet-rich plasma.

25-62. (canceled)
